(19) United States
(12) Patent Application Publication
(10) Pub. No.: US 2016/0175223 A1
(43) Pub. Date: Jun. 23, 2016

(54) ANTI-AGING COMPOSITIONS COMPRISING BILE ACID-FATTY ACID CONJUGATES

(71) Applicant: GALDERM THERAPEUTICS LTD., Tel Aviv (IL)

(72) Inventors: Nava DAYAN, Fairlawn, NJ (US); Allen BAHARAFF, Tel Aviv (IL)

(73) Assignee: GALDERM THERAPEUTICS LTD., Tel Aviv (IL)

(21) Appl. No.: 14/910,477

(22) PCT Filed: Aug. 7, 2014
(86) PCT No.: PCT/IL2014/050717

§ 371 Date: Feb. 5, 2016

(30) Foreign Application Priority Data
Aug. 8, 2013 (IL) 227890

(51) Int. Cl. A61K 8/63; A61Q 19/08

(52) U.S. Cl. CPC A61K 8/63; A61Q 19/08

(57) ABSTRACT

Provided are topical compositions including fatty acid bile acid conjugates (FABACs). Further provided are methods of using the disclosed compositions for preventing, attenuating or treating skin aging and symptoms related thereto.

### FIELD OF THE INVENTION

The present invention relates to topical compositions comprising bile acid-fatty acid conjugates and their use in treatment or prevention of skin conditions relating to aging.

### BACKGROUND OF THE INVENTION

### Skin and Aging

The skin is the largest organ of the body and functions primarily to protect the body from external/environmental factors such as opportunistic pathogenic microorganisms, chemicals, UV radiation as well as to assist with temperature modulation.

Skin is subject to constant insult by the above environmental factors as well as by inherent factors. Environmental factors affecting skin include exposure to the sun, smoking and air pollution, while inherent factors include stress and chronological aging. Chronological aging may be caused by sub-chronic inflammation related to normal cellular oxidative stress.

Whether extrinsic or intrinsic, challenges faced by skin result in visible signs such as fine lines, wrinkles, uneven texture and scattered pigmentation. The prevention, elimination or diminution of these signs has become a multi-billion dollar business with treatments ranging from over-the-counter topical creams and moisturizers to a variety of beautifying plastic surgery techniques. Normal chronological aging results in skin thinning, loss of elasticity and general atrophy of the skin. Chronological aging may be hastened by photo-aging, the premature aging of the skin due to exposure to UV radiation. Oxidation may also contribute to the process of skin aging by producing unstable molecules, known as free radicals, which, when produced in access and/or chronically, may accumulate and damage skin cells.

Western Society is judging elderly looking individuals as less attractive. This is fostering a very significant industry that is supporting the aging population with means to maintain youthful appearance. Premature aging of the skin is associated with wrinkles that can have a profound impact on self-esteem.

Retinoic acid (also known as tretinoin) is currently the only prescribed topical drug with an anti-aging and wrinkle reduction indication. While the effect of retinoic acid on anti-aging marks is significant, especially with reversing wrinkle and damage associated with photo-aging, its use is associated with several major side effects including teratogenicity, primary irritation and photo-sensitivity. Therefore retinoic acid must be used under physician supervision. Retinoids, which are commonly used for anti-aging treatments, have been shown to be cytotoxic for fibroblasts and epithelial cells in the range of 0.6-3×10⁵ M (Varani et al. Journal of Investigative Dermatology (1993) 101, 839-842), and to increase epithelial cell death (Ding et al. Invest Ophthalmol Vis Sci. 2013 Jun. 26; 54(6):4341-50).

Fatty Acid Bile Acid or Bile Salt conjugates (FABACs), referred to also as Bile Acid Fatty Acid conjugates (BAFACs), are a family of synthetic molecules that may be used to improve conditions related to bile acids or cholesterol metabolism. FABACs are believed to lower blood cholesterol concentration, reduce liver fat levels and dissolve gallstones (Gilat et al., Hepatology 2003; 38: 436-442; and Gilat et al., Hepatology 2002; 35: 597-600).

U.S. Pat. Nos. 6,384,024, 6,395,722, 6,589,946 disclose use of certain FABACs in dissolving cholesterol gallstones in bile and treating arteriosclerosis. These and additional FABACs were disclosed in U.S. Pat. No. 7,501,403 and U.S. Pat. No. 8,110,564 as well as in US Application Publication US 2012/0214872 for use in treating fatty liver, in reducing blood cholesterol levels and in treating hyperglycemia, diabetes, insulin resistance and obesity. More recently, US Application No. 2012/0157419 disclosed FABACs as useful for treating brain diseases characterized by amyloid plaque deposits (e.g., Alzheimer's disease).

Canadian Patent Application 2,166,427 discloses use of bile acids chenodeoxycholic acid and/or ursodeoxycholic acid for the preparation of a medicament for treatment of atopic dermatitis. WO02/083147 discloses certain bile acid derivatives as Farnesoid X receptor (FXR) ligands for prevention or treatment of FXR-mediated diseases or conditions. Nowhere in the art is it disclosed or suggested that FABACs may be useful for topical administration, and particularly for prevention, treatment or attenuation of disorders associated with skin aging.

There is an unmet need for compositions and methods useful in treating the symptoms associated with aging of the skin, including but not limited to, wrinkles formation.

### SUMMARY OF THE INVENTION

The present invention relates to skin care, particularly cosmetic compositions comprising fatty acid bile acid conjugates (FABACs) and method of use thereof for preventing, attenuating or treating skin aging and symptoms related thereto.

The present invention is based in part on the unexpected discovery that FABACs are able to reduce gene expression levels of keratin 10 and keratin 1 in skin fibroblasts, as exemplified herein below. According to some embodiments, reduced expression of keratin 1 and 10 results in reduced keratinocyte differentiation.

The present invention is further based on the surprising discovery that administration of FABACs does not result in a significant effect on viability of epidermal cells, in contrast to administration of retinoids.

Furthermore, as exemplified herein below, administration of FABACs results in enhanced efflux of cholesterol into skin fibroblasts. Thus, without wishing to be bound by any theory or mechanism, FABACs may act on lipid rafts in skin cells and attenuate keratinocytes differentiation in a mechanism that is similar to that of retinoic acid but without the serious adverse reactions affiliated with retinoic-acid treatment such as teratogenicity, skin irritation and higher susceptibility to sun damage.

According to a first aspect the present invention provides a topical composition comprising as an active ingredient a fatty acid bile acid conjugate (FABAC) and at least one dermatologically acceptable diluent, carrier or excipient, wherein the FABAC has the formula I:

W—X-G (I)

wherein G represents a bile acid or a bile salt radical; W represents one or two fatty acid radicals having 6-22 carbon atoms; and X represents a bonding member comprising a heteroatom or a direct C—C or C═C bond. Each possibility represents a separate embodiment of the present invention. According to some embodiments, the composition is formulated for topical administration. The term “topical administration”, as used herein, refers to administration through body surfaces, preferably through skin. According to another embodiment, the composition is formulated in a form selected from the group consisting of: aqueous solution, cream, lotion, water in oil or oil in water emulsion, multiple emulsion, silicone emulsion, microemulsion, nanoemulsion, gel, foam and an aqueous solution with a co-solvent. Each possibility represents a separate embodiment of the present invention. According to some embodiments, the topical composition is a cosmetic composition formulated for topical administration.

According to some embodiments, the term “dermatologically acceptable diluent, carrier or excipient” refers to any diluent, carrier or excipient known in the art to be suitable for application to the skin. According to some embodiments, the at least one dermatologically acceptable diluent, carrier or excipient is cosmetically suitable. According to certain embodiments, the at least one dermatologically acceptable diluent, carrier or excipient is pharmaceutically acceptable. According to certain embodiments, the topical composition comprises at least one pharmaceutically acceptable carrier, diluent or excipient suitable for topical administration, preferably suitable for application to the skin.

According to another embodiment, the bonding member is selected from the group consisting of: NH, P, S, O, and a direct C—C or C═C bond. Each possibility represents a separate embodiment of the present invention. According to an exemplary embodiment, said bonding member is NH.

According to some embodiments, the FABAC comprises two fatty acid radicals wherein at each occurrence W is independently a fatty acid radical having 6-22 carbon atoms; and X is independently a bonding member comprising a heteroatom or a direct C—C or C═C bond. Each possibility represents a separate embodiment of the present invention.

According to another embodiment, said one or two fatty acid radicals are independently selected from radicals of a fatty acid selected from the group consisting of: stearic acid, behenic acid, arachidylic acid, palmitic acid, arachidonic acid, eicosapentaenoic acid, oleic acid. Each possibility represents a separate embodiment of the present invention. According to yet another embodiment, said one or two fatty acid radical is a radical of stearic acid.

According to another embodiment, said bile acid is selected from the group consisting of: cholic acid, ursodeoxycholic acid, chenodeoxycholic acid, deoxycholic acid, lithocholic acid, and derivatives thereof. Each possibility represents a separate embodiment of the present invention. According to yet another embodiment, said bile acid is a cholic acid.

According to some embodiments, the term “bile salt radical” as used herein refers to a bile salt radical of a bile acid selected from the group consisting of: cholic acid, ursodeoxycholic acid, chenodeoxycholic acid, deoxycholic acid, lithocholic acid, and derivatives thereof. Each possibility represents a separate embodiment of the present invention. According to some embodiments, the bile salt radical is a bile salt radical of cholic acid.

According to some embodiments, said FABAC is selected from the group consisting of:

3-beta-stearoyl-amido,7α,12α-dihydroxy-5-beta-cholan-24-oic acid;

3-beta arachidylamido,7α,12α-dihydroxy-5-beta-cholan-24-oic acid; and a combination thereof. Each possibility represents a separate embodiment of the present invention.

According to an exemplary embodiment, said FABAC is 3-beta-stearoyl-amido,7α,12α-dihydroxy-5-beta-cholan-24-oic acid (also referred to herein as “Steamchol”).

According to some embodiments, the present invention provides a cosmetic composition comprising at least one FABAC as an active ingredient, preferably Steamchol, wherein the cosmetic composition is formulated for topical administration and further comprises at least one cosmetically acceptable diluent, carrier or excipient suitable for topical administration.

According to another embodiment, the composition of the present invention is useful for preventing or treating a skin condition related to aging. Each possibility represents a separate embodiment of the present invention. According to another embodiment, the composition of the present invention is useful for treating a skin condition related to aging.

According to another aspect, there is provided a method of preventing or treating a skin condition related to aging comprising the step of administering to a subject in need thereof a topical composition, the composition comprising as an active ingredient a fatty acid bile acid conjugate (FABAC) and at least one dermatologically acceptable diluent, carrier or excipient, wherein the FABAC has the formula I:

W—X-G (I)

wherein G represents a bile acid or a bile salt radical; W represents one or two fatty acid radicals having 6-22 carbon atoms; and X represents a bonding member comprising a heteroatom or a direct C—C or C═C bond; thereby preventing or treating the skin condition related to aging. Each possibility represents a separate embodiment of the present invention.

According to some embodiments, there is provided a topical composition for use in preventing or treating a skin condition related to aging, the composition comprising as an active ingredient a fatty acid bile acid conjugate (FABAC) and at least one dermatologically acceptable diluent, carrier or excipient, wherein the FABAC has the formula I:

W—X-G (I)

wherein G represents a bile acid or a bile salt radical; W represents one or two fatty acid radicals having 6-22 carbon atoms; and X represents a bonding member comprising a heteroatom or a direct C—C or C═C bond. Each possibility represents a separate embodiment of the present invention.

According to some embodiments, the present invention provides at least one fatty acid bile acid conjugate (FABAC) for use in preparation of a topical composition for treatment of a skin condition associated with altered sebum levels, wherein the FABAC has the formula I:

W—X-G (I)

wherein G represents a bile acid or a bile salt radical; W represents one or two fatty acid radicals having 6-22 carbon atoms; and X represents a bonding member comprising a heteroatom or a direct C—C or C═C bond, thereby treating the skin conditions associated with altered sebum levels in said subject. Each possibility represents a separate embodiment of the present invention. According to some embodiments, the topical composition further comprises at least one dermatologically acceptable diluent, carrier or excipient. According to some embodiments, the topical composition is a cosmetic composition.

According to another embodiment, the skin condition related to aging is associated with at least one of chronological aging, photo-aging, skin atrophy or a combination thereof. Each possibility represents a separate embodiment of the present invention.

According to some embodiment, the skin condition related to aging is selected from the group consisting of: fine lines, wrinkles, discoloration, uneven pigmentation sagging, enlarged pores, rough skin, dry skin and stretch marks, uneven tone, blemishes, skin thickening or thinning and a combination thereof. Each possibility represents a separate embodiment of the present invention. According to some embodiments, the skin condition related to aging may be any other aging related skin appearance either associated with chronological and/or environmental aging. Each possibility represents a separate embodiment of the present invention.

According to another embodiment, the skin condition related to aging is wrinkling. According to yet another embodiment, the skin condition related to aging is fine lines. According to some embodiments, the skin condition related to aging is selected from the group consisting of: skin wrinkles, skin atrophy, photo-aging and a combination thereof. Each possibility represents a separate embodiment of the present invention.

Other objects, features and advantages of the present invention will become clear from the following description and drawings.

### BRIEF DESCRIPTION OF THE FIGURES

FIG. 1 shows a process for producing 3β-stearylamido-7α,12α, dihidroxy-5β-cholan-24-oic acid (stearyl amido cholanoic acid also referred herein as “Steamchol”), according to some embodiments.

FIG. 2 depicts comparison of viability of epidermal cell cultures which were untreated (Negative Control), treated with 1% Triton X-100 (Positive Control), with DMSO alone, or with DMSO containing either 0.01%, 0.1%, 1% or 2% of Steamchol.

### DETAILED DESCRIPTION OF THE INVENTION

The present invention relates to topical compositions comprising a fatty acid bile acid conjugate (FABAC) useful as an anti-aging agent. As used herein, the term “anti-aging agent” relates to an agent able to treat or prevent at least one skin condition related to aging. The invention further relates to methods of preventing, attenuating or treating skin conditions related to aging and symptoms thereof, including, but not limited to, skin wrinkling, through topical administration of the disclosed composition.

Cholesterol is the second most abundant lipid by weight in the stratum corneum (after ceramides) and is known to promote the intermixing of different lipid species and regulate their thermodynamic “phase behavior”. Keratinocytes require abundant amounts of cholesterol for maintaining a strong barrier and to control cutaneous permeability; hence the regulation of cholesterol homeostasis in the skin is of great importance. ATP-binding cassette transporter (ABCA1) is a membrane transporter for cholesterol efflux playing a pivotal role in regulating cellular cholesterol levels. Lipid rafts existing in cell membranes generally contain 3 to 5-fold the amount of cholesterol found in the surrounding bilayer.

As exemplified herein below, administration of a FABAC compound resulted in elevated levels of ABCA-1 cholesterol transporter in skin cells in parallel to an elevation in cholesterol efflux. Moreover, administration of FABAC was exemplified to significantly down-regulate mRNA levels of keratinocyte differentiation markers keratins 1 and 10 in skin cells.

Without wishing to be bound by any theory or mechanism of action, FABACs show advantageous anti-aging effect by enhancing ABCA1 transporter in fibroblasts and down regulating mRNA levels of the keratinocyte differentiation markers keratins 1 and 10. Thus, FABACs may affect cellular differentiation at epidermal skin layers in a mechanism similar to that of retinoic-acid. As further exemplified below, the mRNA level of ABCA-1 were not elevated as a result of FABAC administration. Thus, without wishing to be bound by theory or mechanism, FABACs may affect differentiation in epidermal skin layers, though without direct effect on nucleus associated retinoic acid receptors. Thus, according to some embodiments, FABAC do not cause the serious adverse reactions affiliated with retinoic-acid treatment such as teratogenicity, skin irritation and higher susceptibility to sun damage.

As exemplified herein below, Steamchol demonstrated very low cellular toxicity in a cytotoxicity assay using a cell culture including fibroblasts and keratinocytes. According to some embodiments, FABCAs, such as, but not limited to, Steamchol, induce no or very limited cell death when topically administered in a composition at a concentration of up to 10% weight volume, possibly up to 5% weight/volume, most typically up to 2% weight/volume. Each possibility represents a separate embodiment of the present invention.

According to one aspect the present invention provides a topical composition comprising as an active ingredient a fatty acid bile acid conjugate (FABAC) and at least one dermatologically acceptable diluent, carrier or excipient, wherein the FABAC has the formula I:

W—X-G (I)

wherein G represents a bile acid or a bile salt radical; W represents one or two fatty acid radicals having 6-22 carbon atoms; and X represents a bonding member comprising a heteroatom or a direct C—C or C═C bond. Each possibility represents a separate embodiment of the present invention.

The term “FABAC” (synonym BAFAC) as used herein, refers to conjugates of the formula W—X-G (Formula I), wherein G represents a bile acid or bile salt radical thereof, W represents one or two fatty acid radical(s) having 6-22 carbon atoms, and X represents a bonding member between said bile acid and the fatty acid radical(s).

According to some embodiments, bonding member X includes, but is not limited to, NH, P, S, O or a direct C═C or C—C bond. Each possibility represents a separate embodiment of the present invention. FABACs are known in the art, and are described, for example, in U.S. Pat. Nos. 6,384,024, 6,395,722, and 6,589,946, the contents of which are incorporated herein by reference. According to some embodiments, the fatty acid radical(s) comprise specify 8-22 carbon atoms, 14-22 carbon atoms or 18-22 carbon atoms. Each possibility represents a separate embodiment of the present invention. As used herein, the terms “FABACs”, “BAFACs”, “the FABACs” and “the FABACs of the invention” are used interchangeably. According to some embodiments, the topical composition of the invention comprises at least one FABAC.

A non-limiting general structure of FABACs is set forth below. According to a non-liming example, bile acid is conjugated (e.g. using an amide bond, for example at position 3) with 1-2 fatty acids of any of a number of chain lengths.

According to an exemplary embodiment, the FABAC of the invention is 3β-arachidylamido-7α,12α, dihidroxy-5β-cholan-24-oic acid (Arachidyl Amido Cholanoic Acid; an amide conjugate of cholic acid with arachidic acid; also known as “Aramchol” or “C20 FABAC”) or 3β-stearylamido-7α,12α, dihidroxy-5β-cholan-24-oic acid (Stearyl Amido Cholanoic Acid; an amide conjugate of cholic acid with stearic acid; also known as “Steamchol” or “C18 FABAC”). Each possibility represents a separate embodiment of the present invention. According to some embodiment, the FABAC is Steamchol.

In another embodiment, the FABAC of methods and compositions of the present invention has the formula I:

W—X-G (I)

wherein G represents a bile acid or a bile salt radical; W represents one or two radicals of saturated or unsaturated fatty acids having 6-22 carbon atoms; and X represents a bonding member comprising a heteroatom or a direct C—C or a C═C bond. Each possibility represents a separate embodiment of the present invention. According to some embodiments, G represents a radical of a bile acid. According to some embodiments, X represents a bonding member selected from the group consisting of: a heteroatom, a direct C—C bond and a C═C bond. Each possibility represents a separate embodiment of the present invention.

According to some embodiments, the FABACs of methods and compositions of the present invention have the formula II:

(W—X—)n G (II)

wherein G represents a bile acid or a bile salt radical; W represents a fatty acid radical having 6-22 carbon atoms; X represents a bonding member comprising a heteroatom or a direct C—C or C═C bond; and n is an integer 1 or 2. Each possibility represents a separate embodiment of the present invention. According to some embodiments, the heteroatom is selected from the group consisting of: NH, P, S and O. Each possibility represents a separate embodiment of the present invention. In general, the term “heteroatom” includes atoms of any element other than carbon or hydrogen, preferred examples of which include nitrogen, oxygen, sulfur, and phosphorus.

According to one embodiment n is 1. According to another embodiment n is 2, and at each occurrence W is independently a fatty acid radical having 6-22 carbon atoms and X is independently a bonding member comprising a heteroatom or a direct C—C or C═C bond. Each possibility represents a separate embodiment of the present invention.

In another embodiment, the bonding member of the FABAC is selected from the group consisting of NH, P, S, O, or a direct C—C or C═C bond. Each possibility represents a separate embodiment of the present invention. According to some embodiments, the term “Direct bond” refers, to a C—C (single) bond. In another embodiment, the term “Direct bond” refers to a C═C (double) bond. In another embodiment, more than one direct bond is utilized in the FABAC of the invention. In another embodiment, the bond between the bile acid and the fatty acid radical(s) is in the beta configuration. In another embodiment, the bond between the bile acid and the fatty acid radical(s) is in the alpha configuration. In another embodiment, the bonding member is other than an ester bond.

According to some embodiments, the bile acid or bile acid radical of the FABAC is selected from the group consisting of: cholic acid, ursodeoxycholic acid, chenodeoxycholic acid, deoxycholic acid, lithocholic acid and derivatives thereof. Each type of bile acid or radical thereof represents a separate embodiment of the present invention. The term “radical” as used herein means a chemical moiety comprising one or more unpaired electrons. According to some embodiments, the bile acid or bile acid radical of the FABAC is cholic acid.

In another embodiment, the FABAC comprises a single fatty acid radical. The conjugation of the bile acid with the fatty acid radical may take place at various positions of the bile acid. In certain embodiments, the conjugation of the bile acid with the fatty acid radical is performed in a position of the bile acid nucleus selected from positions 3, 6, 7, 12 and 24. Each possibility represents a separate embodiment of the present invention. In one embodiment, said conjugation is performed in position 3 of the bile acid nucleus.

In another embodiment, the FABAC comprises two fatty acid radicals. According to some embodiments, the conjugation of each fatty acid radical to the bile acid nucleus is at two positions selected from the 3, 7, 12 and 24 positions of the bile acid nucleus. Each possibility represents a separate embodiment of the present invention. According to a particular embodiment, the conjugations are at position 3 and 7 of the bile acid nucleus.

In another embodiment, the fatty acid is saturated. In another embodiment, the fatty acid is unsaturated. In another embodiment, the fatty acid is mono-unsaturated. In another embodiment, the fatty acid is poly-unsaturated.

In another embodiment, the fatty acid(s) or fatty acid radical(s) of the FABAC are independently selected from the group consisting of: behenic acid, arachidylic acid, stearic acid, and palmitic acid. Each possibility represents a separate embodiment of the present invention.

An exemplary embodiment of a FABAC according to the present invention is presented in Formula III herein below. According to some embodiments, in Formula III n=20 or n=18. Each possibility represents a separate embodiment of the present invention.

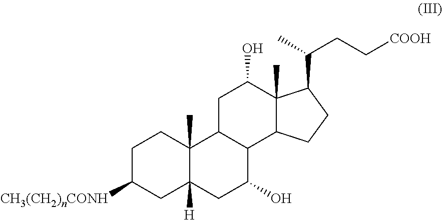

According to some embodiments, the one or two fatty acids or fatty acid radicals of the FABACs of the invention are unsaturated fatty acids or fatty acid radicals. Each possibility represents a separate embodiment of the present invention. In another embodiment, the unsaturated fatty acid(s) or unsaturated fatty acid radical(s) of the FABAC are independently selected from the group consisting of: linolenic acid, eicosapentaenoic acid, docosahexaenoic acid, arachidonic acid, palmipoleic acid, oleic acid and elaidic acid. Each possibility represents a separate embodiment of the present invention. A non-limiting example of FABAC comprising an unsaturated fatty acid is 3β-oleylamido-7α,12α-dihidroxy-5β-cholan-24-oic acid, as depicted in Formula IV herein below.

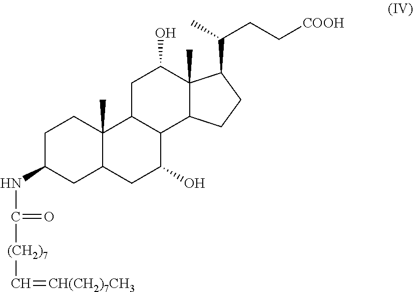

In another embodiment, linoleic acid is utilized. In another embodiment, a conjugated linoleic acid is utilized. In another embodiment, a conjugated linoleic acid isomer is utilized. Each possibility represents a separate embodiment of the present invention. The term “conjugated fatty acid”, also known as “CFA”, refers to polyunsaturated fatty acids in which at least one pair of double bonds are separated by only one single bond.

In another embodiment, the fatty acid is a short-chain fatty acid. In another embodiment, the fatty acid chain length is 6-8 carbons. In another embodiment, the fatty acid is a medium chain fatty acid. In another embodiment, the fatty acid chain length is 8-14 carbons. In another embodiment, the fatty acid chain length is 14-22 carbons. In another embodiment, the fatty acid chain length is 16-22 carbons. In another embodiment, any other fatty acid chain length known in the art is utilized. Each type of fatty acid or fatty acid radical represents a separate embodiment of the present invention.

According to some embodiments, the FABAC of methods and compositions of the present invention is selected from the group consisting of: 3β-behenylamido-7α,12α-dihydroxy-5β-cholan-24-oic acid; 3β-arachidylamido-7α,12α-dihydroxy-5β-cholan-24-oic acid; 3β-stearylamido-7α,12α-dihydroxy-5β-cholan-24-oic acid; 3β-palmitylamido-7α,12α-dihydroxy-5β-cholan-24-oic acid; 3β-myristylamido-7α,12α-dihydroxy-5β-cholan-24-oic acid; and N—(-carboxymethyl)-3β-stearylamido-7α,12α-dihydroxy-5β-cholane-24-amide. Each possibility represents a separate embodiment of the present invention.

According to some embodiments, the FABAC of methods and compositions of the present invention is selected from the group consisting of:

3-beta-stearoyl-amido,7α,12α-dihydroxy-5-beta-cholan-24-oic acid;

3-beta arachidylamido,7α,12α-dihydroxy-5-beta-cholan-24-oic acid;

3-beta arachidonylamido,7α,12α-dihydroxy-5-beta-cholan-24-oic acid; and a combination thereof. Each possibility represents a separate embodiment of the present invention.

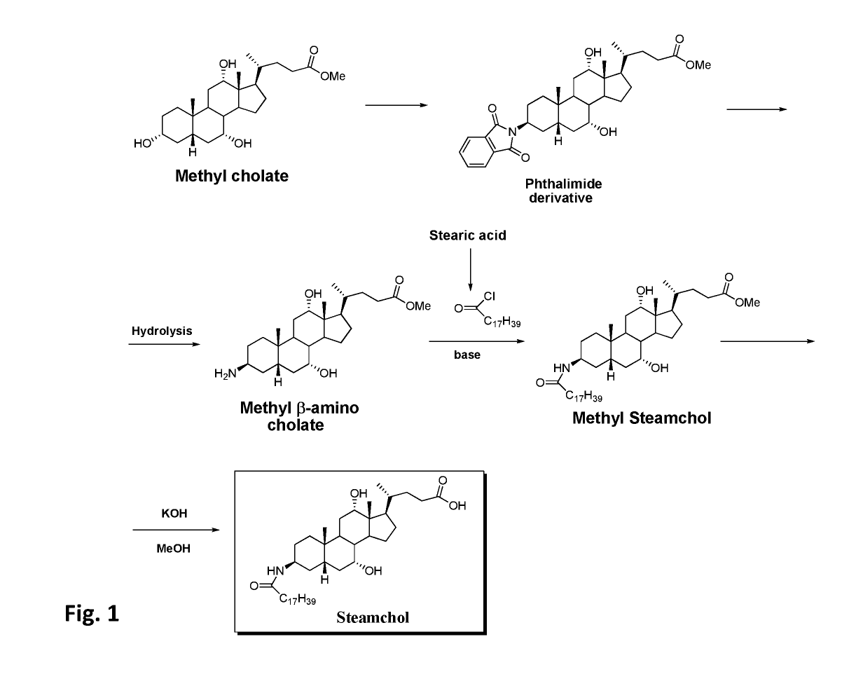

In an exemplary embodiment, the FABAC of the methods and compositions of the present invention is 3β-stearoylamido-7α,12α-dihydroxy-5β-cholan-24-oic acid (“Steamchol”). An exemplary embodiment for producing 3β-stearylamido-7α,12α, dihidroxy-5β-cholan-24-oic acid is presented herein in FIG. 1. According to some embodiments, the present invention provides methods for producing 3β-stearylamido-7α,12α, dihidroxy-5β-cholan-24-oic acid, including but not limited to the process described in FIG. 1.

FABACs as described herein may include pharmaceutically acceptable salts, derivatives and prodrugs. Methods for preparing FABACs and salts, derivatives and prodrugs of FABACs are well known in the art, and are further described in U.S. Pat. No. 6,384,024, 6,395,722 and 6,589,946 and WO 2002/083147, the contents of which are incorporated herein as if set forth in their entirety. As used herein, the term “bile acid derivative” includes bile acid salts with their pharmaceutically acceptable bases or acids as well as their diastereoisomeric and enantiomeric forms.

According to some embodiments, the present invention provides a topical composition comprising at least one of the FABACs of the invention as an active ingredient and at least one diluent, carrier or excipient suitable for topical administration to skin. According to some embodiments, the present invention provides a cosmetic composition formulated for topical administration to skin, comprising at least one of the FABACs of the invention as an active ingredient and at least one diluent, carrier or excipient suitable for topical administration to skin. According to some embodiments, the at least one diluent, carrier or excipient suitable for topical administration is cosmetically acceptable. According to some embodiments, the at least one diluent, carrier or excipient suitable for topical administration is pharmaceutically acceptable.

According to some embodiments, the present invention provides a topical composition comprising Steamchol as an active ingredient and at least one diluent, carrier or excipient suitable for topical administration to skin. According to some embodiments, the disclosed composition is formulated for topical administration to skin, preferably as a cosmetic composition.

According to some embodiments, the present invention provides the disclosed topical composition for preventing or treating a skin condition related to aging. Each possibility represents a separate embodiment of the present invention. According to another embodiment, the composition of the present invention is useful for treating a skin condition related to aging.

Skin renewal is essential for maintaining healthy homeostasis and it is maintained by controlling the balance between proliferation, differentiation and apoptosis of epidermal cells. The program of epidermal differentiation in keratinocytes appears to be altered upon disruption of cholesterol-enriched domains in the plasma membrane. The mechanism for this cholesterol depletion effect was shown to lead to changes in keratinocytes differentiation. The direct correlation between attenuation of keratinocytes differentiation and clinically younger appearing skin is not fully elucidated, however a known compound that demonstrated such activity, retinoic acid, is shown to mitigate and even reverse aging damage. The mechanism may be related to a downstream compensation effect leading to accelerated desmosome cleavage and faster skin renewal as well as affects the extracellular matrix in the dermis.

The present invention is based, in part, on the unexpected finding that fatty acid-bile acid conjugates (FABACs) enhance ABCA1 transporter in fibroblasts and down regulate mRNA levels of the keratinocytes differentiation markers keratins 1 and 10. Without wishing to be bound by any theory or mechanism, these two key effects of FABACs provide the basis for their anti-aging effect.

According to another aspect, the present invention provides a method of preventing or treating a skin condition related to aging comprising the step of administering to a subject in need thereof a dermatologically acceptable amount of a topical composition, the composition comprising as an active ingredient a fatty acid bile acid conjugate (FABAC) and at least one dermatologically acceptable diluent, carrier or excipient, wherein the FABAC has the formula I:

W—X-G (I)

wherein G represents a bile acid or a bile salt radical; W represents one or two fatty acid radicals having 6-22 carbon atoms; and X represents a bonding member comprising a heteroatom or a direct C—C or C═C bond; thereby preventing or treating the skin condition related to aging. Each possibility represents a separate embodiment of the present invention.

According to some embodiments, the present invention provides a topical composition for use in treating or preventing a skin condition related to aging, the composition comprising as an active ingredient a fatty acid bile acid conjugate (FABAC) and at least one dermatologically acceptable diluent, carrier or excipient, wherein the FABAC has the formula I:

W—X-G (I)

wherein G represents a bile acid or a bile salt radical; W represents one or two fatty acid radicals having 6-22 carbon atoms; and X represents a bonding member comprising a heteroatom or a direct C—C or C═C bond. Each possibility represents a separate embodiment of the present invention. According to some embodiments, the FABAC is Steamchol.

According to some embodiments, the present invention provides a topical composition, preferably a cosmetic topical composition, for use in treating or preventing a skin condition related to aging, comprising at least one FABAC as the active ingredient. According to some embodiments, the present invention provides a topical cosmetic composition for use in treating or preventing a skin condition related to aging comprising Steamchol as the active ingredient.

According to some embodiments, the present invention provides a topical composition, preferably a cosmetic topical composition, for use in treating or preventing skin wrinkles, comprising at least one FABAC as the active ingredient. According to some embodiments, the present invention provides a topical cosmetic composition for use in treating or preventing skin wrinkles comprising Steamchol as the active ingredient.

According to some embodiments, the present invention provides use at least one FABAC of the invention for preparation of a topical composition for treatment or prevention of a skin condition related to aging. Each possibility represents a separate embodiment of the present invention. According to some embodiments, the topical composition further comprises at least one dermatologically acceptable carrier, diluent or excipient, preferably a cosmetically acceptable carrier, diluent or excipient.

The terms “cosmetically acceptable/suitable” and “dermatologically acceptable/suitable”, as used herein, relate to elements suitable to come into contact with the skin or human skin appendages without posing a risk of toxicity, intolerance, instability, allergic reaction, and the like. According to some embodiments, the cosmetically or dermatologically acceptable ingredients, such as carriers, diluents and excipients, are those capable of being commingled with anti-aging (e.g., anti-wrinkle) active ingredients such as, but not limited to, the FABACs of the invention such that the cosmetically or dermatologically acceptable ingredients and the active ingredients do not interact in a way which would substantially reduce the efficacy of the active ingredients for treating a condition related to skin aging.

As used herein, the terms “effective amount” and “dermatologically effective amount” relate to an amount of compound or a composition that is capable of inhibiting, reducing, attenuating or treating at least part of the symptoms of a skin condition related to aging. According to some embodiments, a dermatologically effective amount of a composition relates to an amount sufficient for inhibiting, reducing, attenuating or treating at least part of the symptoms of a skin condition related to aging upon topical administration of the composition to the skin of a subject in need thereof. Each possibility represents a separate embodiment of the present invention.

The specific dose of a compound administered according to this invention will, of course, be determined by the particular circumstances surrounding the case including, for example, the compound administered, the route of administration, the physiological state of the subject, and the severity of the pathological condition being treated. According to a typical embodiment, the disclosed composition is administered in several dosages over a prolonged period of time until a sufficient response has been achieved, such as, but not limited to attenuation or treatment of symptoms of a skin condition related to aging.

According to some embodiments, the composition of the present invention is configured to be topically administered to a subject, preferably by direct application to the skin of a subject. Each possibility represents a separate embodiment of the present invention. In particular embodiments, the subject is a mammal, preferably a human.

Human skin, as a primary protective barrier, protects the vital organs of the body from external insult such as changes in temperature and humidity, ultraviolet rays and contaminants, and plays an important role in the regulation of biological homeostasis such as thermoregulation. However, as skin ages, it shows skin aging signs such as loss of elasticity, keratinization, formation of skin wrinkles and skin contraction. The cause of this skin aging can be classified as internal factors such as cell gene transformation and cell tissue change, and external factors such as ultraviolet (UV) and humidity. Skin aging effect due to UV is termed “photo-aging”. In photo-aging, oxygen free radicals are generated in cells by UV light. The oxygen free radicals in turn accelerate the synthesis of fiber degrading proteases (such as MMP-1, MMP-3, MMP-9, etc.), enzymes that catabolize proteins such as collagen or elastin that form the elasticity-controlling fibers of the skin's foundation. By way of signal transduction systems, the effects of the free radicals may induce an inflammatory reaction, thereby decreasing the elasticity of the dermal layer and producing skin wrinkles.

As used herein, “skin aging” or “a skin condition related to aging” refers to skin conditions associated with aged skin. According to some embodiments, conditions associated with aged skin in a subject are conditions that may be treated or attenuated by reduction of keratinocyte differentiation in the subject's skin. Non-limiting examples of conditions associated with aged skin include, but are not limited to, wrinkles, sun damage, dull appearance of the skin, sagging skin, jowls, keratosis, melasma, and uneven hyperpigmentation. Each possibility represents a separate embodiment of the present invention. According to some embodiments, a method for treating skin aging comprises treating the skin with an effective amount of a topical composition comprising a FABAC compound as defined above. According to some embodiments, the skin condition related to aging is skin wrinkles According to some embodiments, the skin condition related to aging is skin atrophy.

According to some embodiments, the skin condition related to aging is a skin condition that may be treated and/or attenuated and/or prevented by a reduction in keratinocyte differentiation. Each possibility represents a separate embodiment of the present invention. According to some embodiments, the composition of the invention induces a reduction in keratinocyte differentiation by reducing expression of keratin 1 and/or keratin 10, by enhancing ABCA1 activity or a combination thereof. Each possibility represents a separate embodiment of the present invention.

According to some embodiments, the present invention provides a method of treating, attenuating and preventing skin wrinkles in a subject, comprising topically administering to the subject's skin a topical composition comprising at least one FABAC of the invention as an active ingredient and at least one dermatologically acceptable carrier, diluent or excipient. According to some embodiments, the present invention provides a method of treating, attenuating and preventing skin wrinkles in a subject, comprising topically administering to the subject's skin a topical composition comprising Steamchol as an active ingredient and at least one dermatologically acceptable carrier, diluent or excipient.

In some embodiments the composition of the invention further comprises at least one additional active ingredient other than the FABACs of the invention including, but not limited to, an anti-aging agent. Non-limiting examples of additional active ingredients that may be added to the composition of the invention, include, but are not limited to, retinoic acid and its derivatives, alpha and beta hydroxy acids (e.g., glycolic acid), peptides, anti-oxidants, skin brightening compounds and the like. Each possibility represents a separate embodiment of the present invention.

According to some embodiments, the amount of topical composition and frequency of treatment administered to a subject afflicted with skin wrinkles varies widely depending upon the level of wrinkling already in existence in the subject, the rate of further wrinkle formation, and the level of regulation desired.

The present invention provides, in some embodiments, a method for preventing, retarding, arresting, or reversing atrophy in mammalian skin comprising the step of topically applying to the skin the topical composition of the invention. Each possibility represents a separate embodiment of the present invention. According to some embodiments, the present invention provides a topical composition for treating, preventing, retarding, arresting, or reversing skin atrophy in a subject in need thereof, the composition comprising at least one of the FABACs of the invention as an active ingredient and further comprising at least one dermatologically acceptable carrier, diluent or excipient. Each possibility represents a separate embodiment of the present invention.

As used herein, “atrophy” of skin means the thinning and/or general degradation of the dermis often characterized by a decrease in collagen and/or elastin as well as decreased number and size of fibroblast cells due to reduction in mitosis and access of cells in senescence. Skin atrophy is a natural result of menopause, chronological aging and of photo-aging and often is an undesirable side effect resulting from corticosteroid treatment. Menopause may be physiological menopause or surgery- or treatment-induced menopause.

The present disclosure further provides, according to some embodiments, a method for treating, preventing, attenuating or ameliorating photo-aging or at least part of the symptoms thereof, comprising the step of topically administering the composition of the invention to the skin of a subject afflicted with photo-aging. Each possibility represents a separate embodiment of the present invention. According to some embodiments, the skin condition related to aging is photo-aging. According to some embodiments, the present invention provides the topical composition of the invention for treating, preventing, attenuating or ameliorating photo-aging or at least part of the symptoms thereof. Each possibility represents a separate embodiment of the present invention. As used herein, the term “photo-aging” includes, without limitation, aging of the skin associated with exposure to the sun or other ultraviolet energy sources. Symptoms of photo-aging include, for example, solar lentigo (age spots), solar keratoses dermatoheliosis and combinations thereof. Each possibility represents a separate embodiment of the present invention. The method of treating photo-aging includes, according to some embodiments, topically administering to an individual in need thereof a composition comprising a FABAC compound as defined above.

Herein, the term “treating” includes abrogating, substantially inhibiting, slowing or reversing the progression, substantially ameliorating clinical symptoms, or substantially preventing the appearance of symptoms associated with a skin condition related to aging, such as, but not limited to, skin wrinkles, photo-aging and skin atrophy. According to some embodiments, the term “treating” is further meant to include improvement of skin appearance and texture, improvement of skin hydration, healing, smoothing of the skin or any combination thereof. Each possibility represents a separate embodiment of the present invention. According to some embodiments, the term “treating” refers to at least partial smoothing of existing wrinkles and/or slowing of deepening of existing wrinkles and/or preventing formation of new wrinkles. Each possibility represents a separate embodiment of the present invention. According to some embodiments, the term “treating” refers to amelioration, arrest or prevention of skin thinning and/or skin degradation. Each possibility represents a separate embodiment of the present invention. According to some embodiments, the term “treating” refers to amelioration, arrest or prevention of photo-aging or at least part of the symptoms thereof. Each possibility represents a separate embodiment of the present invention.

The symptoms of a skin condition related to aging may include, but are not limited to: fine lines, wrinkling, age spots and other discolorations of the skin, sagging skin, growths, dry skin, rough skin, dull skin, acne, alopecia, stretch marks and combinations thereof. Each possibility represents a separate embodiment of the present invention.

According to some embodiments, the treatment of a skin condition related to aging such as skin atrophy or photo-aging of the epidermal cells includes treatment of at least part of the symptoms described herein above with respect to aging of epidermal cells. This treatment may further include prevention of at least part of these symptoms, and in particular aging signs, before they occur. As used herein, the term “preventing” may relate to inhibiting appearance of a skin condition related to aging or at least part of its symptoms. Alternatively, the term “preventing” may relate to inhibiting worsening of existing skin conditions related to aging, such as, but not limited to, worsening of existing skin wrinkles.

According to some embodiments, the topical composition of the invention is formulated for application to the skin of a subject in need thereof. According to a non-limiting example, one method of treating the skin of a subject afflicted with symptoms of a skin condition related to aging, such as wrinkles, is via topical application of a safe amount of the topical composition of the invention. According to some embodiments, symptoms of a skin condition related to aging include, but are not limited to: wrinkles, reduction in skin smoothness, non-even skin tone, impaired skin complexion and the like. The frequency of topical application to the skin may vary widely, depending upon personal needs, but it is suggested as a non-limiting example that topical application of the composition of the invention will range from about once per week to about 10 times daily, preferably from about twice per week to about 4 times daily, more preferably from about 3 times a week to about twice daily, most preferably about once per day. Each possibility represents a separate embodiment of the present invention. According to some embodiments, the topical application would preferably be over a period of from about one month to several years.

“Skin”, as used herein, refers to any epidermal surface prone to aging and can also include, without limitation, the surface of the face and neck, hands, elbows, upper arm region, knees, thighs, legs, feet, breasts, chest, stomach, buttocks, and back area. Preferably, the term “skin” refers to the surface of the face and neck.

According to some embodiments, the present invention provides a skin care treatment method for treating the cutaneous signs of aging, such as, but not limited to, wrinkles and skin atrophy and/or for protecting the skin against the harmful effects caused by ultraviolet (UV) radiation, the method comprising: topically applying, to skin or skin appendages to be treated, the topical composition of the present invention.

In some embodiments there is provided a method for slowing the aging process of the human skin, reducing the signs of aging of the human skin or both, the method comprising applying to the skin of a subject afflicted with skin aging the topical composition of the invention. Slowing the aging process of the human skin and reducing the signs of aging of the human skin may include, but is not limited to, improvement of the skin tone, elasticity or contraction, reduction of wrinkles, removal of lines, combating the formation of skin wrinkles, promotion of skin firmness, reduction of skin sensitivity and irritability or any combination thereof. Each possibility represents a separate embodiment of the present invention.

In additional embodiments there is provided a method for protecting and/or improving the state of the skin of a subject and/or treating imperfections of the skin of a subject in need thereof, the method comprising topically administering the composition of the invention to the skin of a subject. In additional embodiments, there is provided a method for protecting the skin of a subject from skin conditions related to aging, comprising the step of administering the topical composition of the invention to the skin of the subject. According to some embodiment, protecting the skin of the subject relates to prevention of further worsening of existing skin conditions related to aging and/or arrest or slowing of existing skin conditions related to aging or symptoms thereof. Each possibility represents a separate embodiment of the present invention.

### Formulations

According to some embodiments, the topical composition of the invention is formulated as a cosmetic composition comprising at least one FABAC as an active agent. According to some embodiments, the topical composition is formulated for topical administration to the skin of the subject, preferably to skin areas affected by skin conditions associated with aging such as, but not limited to, wrinkled skin or skin affected by photo-aging. As used herein, the term “topical composition” refers to a composition formulated for topical administration to skin.

The compositions of the present invention may be manufactured by processes well known in the art, e.g., by means of conventional mixing, dissolving, granulating, dragger-making, levigating, emulsifying, encapsulating, entrapping or lyophilizing processes.

The compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

According to some embodiments, the skin treatment compositions of the invention comprise a dermatologically or cosmetically acceptable carrier to act as a diluent, dispersant or vehicle for at least one FABAC, so as to facilitate its distribution when the composition is applied to the skin. Vehicles other than, or in addition to, water may include liquid or solid emollients, solvents, humectants, thickeners and powders. Each possibility represents a separate embodiment of the present invention.

According to some embodiments, the composition of the present invention may be formulated for topical administration in the form of aqueous or non-aqueous solutions, lotions, creams, gels, ointments, foam, mousse, sprays, emulsions, microemulsions, adhesive patches, powders etc. Each possibility represents a separate embodiment of the present invention. The formulation may be oleaginous-based, occlusive composition comprising, for example, white petroleum and or mineral oil. In some embodiments the composition is non-greasy or substantially non-greasy and can be a water-based formulation.

According to some embodiments, there is provided a skin care, cosmetic or dermopharmaceutical composition comprising at least one of the FABACs of the invention or a cosmetic, dermatological or pharmaceutically-acceptable salts and esters thereof, and a cosmetically or dermatologically acceptable diluent, carrier or excipient. Each possibility represents a separate embodiment of the present invention.

According to some embodiments, the composition of the invention comprises at least one FABAC. According to some embodiments, the FABAC in the composition of the invention is in an effective amount sufficient to treat, ameliorate, slow down or prevent a skin condition related to aging or at least part of the symptoms thereof. Each possibility represents a separate embodiment of the present invention. According to some embodiments, the effective amount is an amount which induces no or non-significant cytotoxicity in skin cells. According to some embodiments, the FABAC in the composition of the invention is in an effective amount sufficient for treating, attenuating, slowing progression or preventing skin wrinkles and/or skin atrophy and/or photo-aging. Each possibility represents a separate embodiment of the present invention. As used here, the terms “effective amount” and “effective concentration” are used interchangeably.

According to some embodiments, the effective concentration of the FABAC in the composition is between about, 0.01 to 10% weight/volume, possibly between 0.01 to 5% weight/volume, alternatively between 0.05 to 2% weight/volume FABAC. Each possibility represents a separate embodiment of the present invention. According to typical embodiments, the effectiveness of the composition also depends on the vehicle (i.e., carrier) and its interaction with the stratum corneum.

According to some embodiments, the weight/volume concentration of FABAC in the composition of the invention is 0.01%-2%, possibly 0.1%-2%, alternatively 1%-2%. Each possibility represents a separate embodiment of the present invention. According to some embodiments, the weight/volume concentration of FABAC in the composition of the invention is at least 0.01%. According to some embodiments, the FABAC in the composition of the invention is Aramchol. According to some embodiments, the FABAC in the composition of the invention is Steamchol.

In some embodiments the composition of the invention further comprises at least one additional active ingredient other than the FABACs of the invention. Non-limiting examples of such active ingredients include, but are not limited to, the following classes of ingredients: vegetable extracts, oil ingredients, whitening agents, anti-oxidants, coloring agents, healing agents, anti-aging agents, anti-wrinkle agents, soothing agents, anti-radical agents, anti-UV agents (or UV absorbers), agents stimulating the synthesis of dermal macromolecules or the skin's energy metabolism (e.g., skin nutrients), hydrating agents, anti-bacterial agents, anti-fungal agents, anti-inflammatory agents, anesthetic agents, agents modulating cutaneous differentiation, pigmentation or depigmentation, agents stimulating nail or hair growth, combinations thereof etc. Each possibility represents a separate embodiment of the present invention.

According to some embodiments, the at least one additional active ingredient is selected from the group consisting of: metal sequestering agents, medicinal agents, whitening agents, sugars and a combination thereof. Each possibility represents a separate embodiment of the present invention.

According to some embodiments, the at least one additional active ingredient is selected from the group consisting of: metal sequestering agents including, but not limited, to disodium edetate, trisodium edetate, sodium citrate, sodium polyphosphate, sodium metaphosphate and gluconic acid; medicinal agents including but not limited to caffeine, tannin, verapamyl, tranexamic acid and derivatives thereof, grabridin, various herbal medicines, tocopherol acetate, glycyrrhizic acid and the derivatives and salts thereof; whitening agent including but not limited to vitamin C, magnesium ascorbic phosphate, glucoside ascorbate, arbutin and kojic acid; sugars including but not limited to glucose, fructose, mannose, sucrose and trehalose. Each possibility represents a separate embodiment of the present invention.

The compositions of the present invention may be present in the forms of aqueous or hydro alcoholic solution, solubilized systems, emulsions, powders, oils, aqueous or anhydrous gels, serum, foam, ointments, aerosols, water-oil two-phase systems, water-oil-powder three-phase systems, etc. Each possibility represents a separate embodiment of the present invention. In some embodiments the composition is applied in a form selected from the group consisting of: a facial cleanser, spray, salve, ointment, lotions, emulsions, creams, gels, essences (beauty lotions), packs patches and masks. Each possibility represents a separate embodiment of the present invention.

In other embodiments, such as in the case of makeup cosmetics, the composition may be used with a wide range of types of cosmetics such as foundations. In additional embodiments, the composition is applied in the form of a toiletry product, e.g., body soap, facial soap, etc. According to some embodiments, the composition may be formulated as a quasi-drug. Further, in the case of quasidrugs, the composition may be formulated for a wide range of applications such as various ointments. The types or forms of the anti-aging agent of the present invention are not limited to these forms and types.

The types or forms of the compositions of the present invention are not limited to these forms and types. In any case, the person skilled in the art will ensure that these additives, the amounts thereof and the selected formulation are selected so as not to be detrimental to the desired, advantageous properties of the composition according to the invention.

According to another embodiment, the composition is formulated as a topical formulation in a form selected from the group consisting of: aqueous solution, cream, lotion, water in oil or oil in water emulsion, multiple emulsion, silicone emulsion, microemulsion, nanoemulsion, gel, foam and an aqueous solution with a co-solvent. Each possibility represents a separate embodiment of the present invention.

Non-limiting examples of suitable topical formulations of the disclosed composition are as follows:

### Lotions and Creams

According to some embodiments, the topical composition of the invention is formulated as a lotion. The lotions contain an effective concentration of one or more FABAC compound as described herein. The compositions of the present invention may also include at least one or more emollient, which can function as either or both a lubricating and thickening agent. The emollients can comprise in total from about 0.1% to about 50%, preferably from about 1% to about 10%, by weight of the composition. Any emollients known to those of skill in the art as suitable for application to human skin may be used. These include, but are not limited to: hydrocarbon oils and waxes, including mineral oil, petrolatum, paraffin, ceresin, ozokerite, microcrystalline wax, polyethylene, and perhydrosqualene; silicone oils; triglyceride fats and oils, including those derived from vegetable, animal and marine source; including jojoba oil and shea butter; acetoglyceride esters, such as acetylated monoglycerides; ethoxylated glycerides, such as ethoxylated glyceryl monostearate; fatty acids, fatty alcohols and derivatives thereof. Each possibility represents a separate embodiment of the present invention. Other suitable emollients include lanolin and lanolin derivatives; polyhydric alcohols and poly ether derivatives; polyhydric alcohol esters; wax esters; vegetable waxes; phospholipids, such as lecithin and derivatives; sterols, including, but not limited to, cholesterol and cholesterol fatty acid esters; amides, such as fatty acid amides, ethoxylated fatty acid amides, and solid fatty acid alkanolamides. Each possibility represents a separate embodiment of the present invention.

The lotions may further contain from about 1% to about 10%, more preferably from 2% to 5%, of an emulsifier. Each possibility represents a separate embodiment of the present invention. The emulsifiers may be nonionic, anionic, cationic or a mixture thereof. Each possibility represents a separate embodiment of the present invention. Suitable emulsifiers are known to those with skill in the art. Other conventional components of such lotions and creams may be included. One such additive is a thickening agent at a level from 1% to 10% of the composition. Examples of suitable thickening agents include, but are not limited to: cross-linked carboxypolymethylene polymers, ethyl cellulose, polyethylene glycols, gum tragacanth, gum karaya, xanthan gums, bentonite and other clays, hydroxy ethyl cellulose, and hydroxypropyl cellulose. Each possibility represents a separate embodiment of the present invention.

According to some embodiments, the lotions and creams are formulated by simply admixing all of the components together. According to some embodiments, the FABAC is dissolved, suspended or otherwise uniformly dispersed in the mixture.

### Solutions and Suspensions

According to some embodiments, the composition is formulated as a solution. According to some embodiments, the composition is formulated as a suspension. According to some embodiments, the solutions, which may be aqueous or non-aqueous, are formulated to contain an effective concentration of one or more FABAC compound as disclosed herein.

Suitable organic materials which may be useful as the solvent or a part of a solvent system in the solution are as follows: propylene glycol, polyethylene glycol, polypropylene glycol, glycerin, sorbitol esters, 1,2,6-hexanetriol, ethanol, isopropanol, diethyl tartrate, butanediol, and mixtures thereof. Each possibility represents a separate embodiment of the present invention. Such solvent systems can also contain water.

According to some embodiments, the composition is formulated as an emulsion. When the compositions of the invention are formulated as an emulsion, the proportion of the fatty phase may range from about 5% to about 80% by weight, and preferably from about 5% to about 50% by weight, relative to the total weight of the composition. Each possibility represents a separate embodiment of the present invention. Oils, emulsifiers and co-emulsifiers incorporated in the composition in emulsion form are selected from among those known to those with skill in the cosmetic or dermatological field.

The compositions formulated as solutions or suspensions may be applied directly to the skin, or, may be formulated as an aerosol and applied to the skin as a spray, foam or mousse. Each possibility represents a separate embodiment of the present invention. The aerosol compositions may further contain from about 20% to 80%, preferably from 30% to 50%, of a suitable propellant. Each possibility represents a separate embodiment of the present invention. Examples of such propellants may be, but are not limited to, the chlorinated, fluorinated and chlorofluorinated lower molecular weight hydrocarbons. Nitrous oxide, carbon dioxide, butane, and propane may also be used as propellant gases. These propellants are used as known in the art in a quantity and under a pressure suitable to expel the contents of the container. In the case of a pressurized aerosol, the dosage unit may be determined by providing a valve to deliver a metered amount.

### Gels and Solids

According to some embodiments, the composition is formulated as a gel. Gel compositions may be formulated by simply admixing a suitable thickening agent to the previously described solution or suspension compositions. Examples of suitable thickening agents have been previously described with respect to the lotions. According to some embodiments, the gelled compositions contain an effective concentration of at least one FABABC compound. According to some embodiments, the composition further comprises from about 5% to about 75% of an organic solvent as previously described; from about 0.5% to about 20% of a thickening agent, and the balance being water or other aqueous carrier.

In other embodiments the compositions formulated as solutions, suspensions lotions and gels of the present invention are formulated as a foam or mousse for dermal application. Each possibility represents a separate embodiment of the present invention. Relevant carriers for formulation as a foam or mousse are taught, for example, in International Patent Application Publication No. WO 2004/037225 and U.S. Pat. No. 6,730,288.

According to some embodiments, the composition is formulated as a solid form. Compositions of solid forms may be formulated as stick-type compositions intended for application to the lips or other parts of the body. The solids may also contain from about 50% to about 98% of the previously described emollients. This composition may contain from about 1% to about 20%, of a suitable thickening agent, and, if desired or needed, emulsifiers and water or buffers. Thickening agents previously described with respect to lotions are suitably employed in the compositions in solid form. Other ingredients, such as preservatives, including methyl-paraben or ethyl-paraben, perfumes, dyes or the like, that are known in the art to provide desirable stability, fragrance or color, or other desirable properties, to compositions for application to the skin.

According to some embodiments, the composition of the present invention is effective to prevent and/or treat skin disorders associated with aging including, but not limited to, reduction in skin elasticity, generation of wrinkles, skin discoloration, skin sagging due to cutaneous aging caused by normal and photo-aging, and combinations thereof. Each possibility represents a separate embodiment of the present invention.

### Additives

According to some embodiments, the composition of the present invention further comprises at least one additive selected from the group consisting of: a diluent, a preservative, an abrasive, an anticaking agent, an antistatic agent, a binder, a buffer, a dispersant, an emollient, an emulsifier, a co-emulsifiers, a humectant or emollient agent, a fiberous material, a film forming agent, a fixative, a foaming agent, a foam stabilizer, a foam booster, a gellant, a lubricant, a moisture barrier agent, a plasticizer, a preservative, a propellant, a stabilizer, a surfactant, a suspending agent, a thickener, a chelating agent, a sequestering agent, a conditioning agent, a wetting agent, a liquefier and a combination thereof. Each possibility represents a separate embodiment of the present invention.

For any agent, combination of agents and composition used within the scope of the invention, the dermatologically effective amount or dose can be estimated initially from cell culture assays. For example, a dose can be formulated in animal models to achieve a circulating concentration range that includes the IC₅₀ as determined in cell culture (e.g., the concentration of the test compound, which achieves a half-maximal inhibition of the epidermal cells proliferation). Such information can be used to more accurately determine useful doses in humans.

Other examples of additives may include sunscreen agents and tanning agents. Sunscreen agents may include those materials commonly employed to block ultraviolet light. Illustrative compounds are the derivatives of PABA, cinnamate and salicylate. For example, octyl methoxycinnamate and 2-hydroxy-4-methoxy benzophenone (also known as oxybenzone) can be used. Octyl methoxycinnamate and 2-hydroxy-4-methoxy benzophenone are also known as parsol MCX and benzophenone-3, respectively. The amount of sunscreen agents employed in the compositions can vary depending upon the degree of UV radiation protection desired. The sunscreen agent added to the composition must be compatible with the active compound but in general the composition may comprise from about 1% to about 20%, of a sunscreen agent. Exact amounts will vary depending upon the sunscreen agent chosen and the desired Sun Protection Factor (SPF).

The composition of the present invention may further comprise an anti-oxidant/radical scavenger. The inclusion of an anti-oxidant/radical scavenger may increase the benefits of the composition. The anti-oxidant/radical scavenger may be added to the compositions of the present invention in a concentration range of about 0.1% to about 10% total weight of the composition. Anti-oxidants/radical scavengers include, but are not limited to, ascorbic acid (vitamin C) and its salts, and tocopherol (vitamin E).

Certain vitamin A metabolites, as well as agonists, derivatives and pro-drugs of vitamin A, may be incorporated into the compositions of the present invention. Examples of vitamin A agents that are useful in the context of the present invention include, without limitation, the well-known variety of retinol, retinoic acid and retinoic acid receptor (RAR) agonists. Each possibility represents a separate embodiment of the present invention. RAR agonists may include, without limitation, chromans, thiochromans, tetrahydroquinolines, substituted tetrahydronaphthalenes, substituted dihydronaphthalenes, trisubstituted phenyls, aromatic tetracyclic compounds, substituted cyclohexanes, substituted cyclohexenes, substituted cyclohexanedienoic acids, substituted adamentanes, substituted diaryl, heteroaryl compounds, combinations thereof and many more. Each possibility represents a separate embodiment of the present invention.

Vitamin C, or ascorbic acid is a very potent antioxidant and may even be protective against UVA and UVB rays. Studies suggest that topical vitamin E, particularly alpha tocopherol (a form of vitamin E) cream decreased skin roughness, length of facial lines, and wrinkle depth. Studies on mice have also reported reductions in UV-induced skin cancer with its use. Vitamin K may also be useful for treating capillary damage. According to some embodiments, the composition further includes at least one of: vitamin C, vitamin E, vitamin K and any combination thereof. Each possibility represents a separate embodiment of the present invention.

Green and black tea and extracts thereof are suitable as additives. Other plant derived agents which may be used as additives to the composition include, but are not limited to, pomegranate and soy extracts, aloe, ginger, grape seed extract, and coral extracts.

Color correctors and foundations are suitable additives and may be desired when blemishes are prominent or when a more even tone of skin is desired. For example, green neutralizers may mask red lesions; yellow may camouflage dark circles and bruises; and white may help to minimize apparent wrinkles. Liquid and press powder foundations may also be included. Other possible additives include glycosaminoglycans, such as hyaluronic acid and the like.

### Product Packaging and Kits

In use, a small quantity of the composition, for example from about 0.1 ml to about 100 ml, is applied to exposed areas of the skin, from a suitable container or applicator and, if necessary, it is then spread over and/or rubbed into the skin using the hand, fingers or a suitable device. The product may be specifically formulated for use as a hand or facial treatment.

When formulated the composition can be packaged in a suitable container to suit its viscosity and intended use by the consumer. For example, a lotion or cream can be packaged in a bottle, or a propellant-driven aerosol device or a container fitted with a pump suitable for finger operation. When the composition is a cream, it can simply be stored in a non-deformable bottle or squeeze container, such as a tube or a lidded jar. The invention accordingly also provides, according to some embodiments, a closed container containing a dermatologically or cosmetically acceptable composition as herein defined. The shape of the container is not limited in this invention, and can be a tube, a pump dispenser, a compressed dispenser, a bottle, a spray, a sachet or the like.

In some embodiments, the compounds or compositions of the invention are provided in packs in a form ready for administration. In other embodiments, the compounds or compositions are provided in concentrated form in packs, optionally with the diluent required to make final solution(s) for administration. In still other embodiments, the product contains a compound useful in the invention in solid form and, optionally, a separate container with a suitable solvent or carrier for the compound useful in the invention.

According to some embodiments, the present invention provides a kit comprising the composition of the invention in a first suitable container. According to some embodiments, the present invention provides a kit comprising at least one FABAC of the invention in a first suitable container. According to some embodiments, the kit further comprises at least one container other than the first container. According to some embodiments, said at least one other container comprises at least one diluent, excipient, carrier, solvent or additive. Each possibility represents a separate embodiment of the present invention. According to some embodiments, the composition of the invention is formed by combining the content of a first container comprising at least one FABAC and the content of said at least one other container. According to some embodiments, the kit further comprises instructions for use and/or preparation of the composition of the invention. Each possibility represents a separate embodiment of the present invention.

In still other embodiments, the above packs/kits include other components, e.g., instructions for dilution, mixing and/or administration of the product, other containers, syringes, needles, etc. Each possibility represents a separate embodiment of the present invention. Other such pack/kit components will be readily apparent to one of skill in the art.

The following examples are presented in order to more fully illustrate some embodiments of the invention. They should, in no way be construed, however, as limiting the broad scope of the invention.

### Examples

### Example 1

### Cytotoxicity Assay Study Overview

Full-thickness Epiderm cultures (EFT-400, MatTek) were used to determine toxicity in response to topical application of a test material (Steamchol). A previous viability assay was conducted at concentrations of 40.0, 13.3, 4.4, 1.5 ug/mL and vehicle alone (DMSO); no toxicity was observed.

Experimental Procedures

The test material was provided as a powder. Test solutions were prepared by adding the appropriate amount of powdered material to 1 mL DMSO. Lower concentrations were made by serial dilution with 100% DMSO. The final test material concentrations and treatments are listed below:
2% Steamchol (20,000 ug/mL)
1% Steamchol (10,000 ug/mL)
0.1% Steamchol (1000 ug/mL)
0.01% Steamchol (100 ug/mL)
vehicle control (100% DMSO)
untreated control

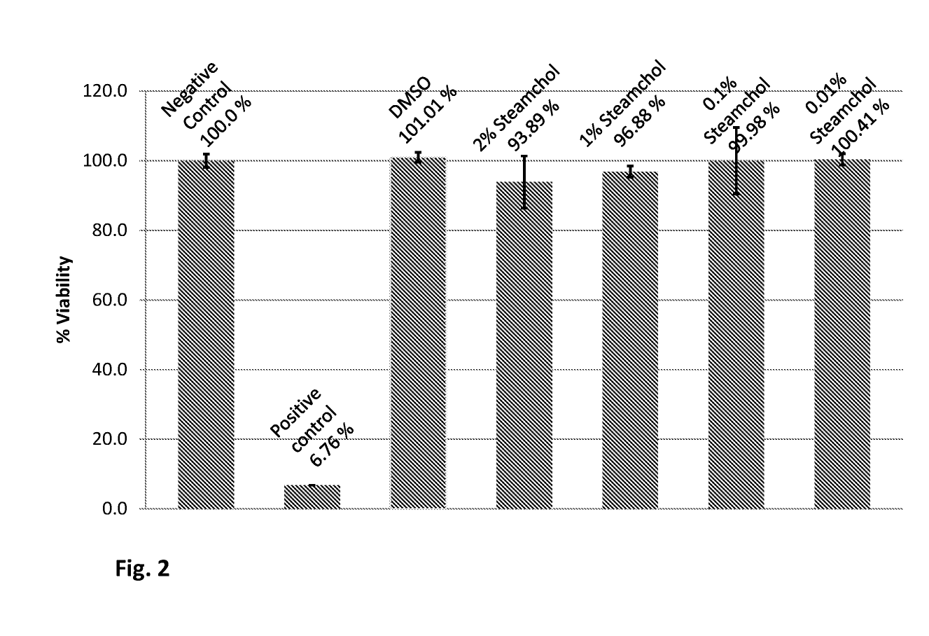

Full-thickness skin cultures (EFT-400) were maintained as required to ensure viability; the cultures were equilibrated at 37° C. and 5% CO₂ for 24 hours prior to application of test materials. Test material was applied to the surface of each culture using sterile techniques (10 μL applied to each culture). Two cultures were assigned to each treatment group. Two EFT cultures served as an untreated control (also referred to as negative control in FIG. 2), and two other EFT cultures served as a positive control (100 μL 1% Triton X-100).

Following a 24-hour incubation period with the test material, cultures were processed for cell toxicity analysis using an MTT [(3-4,5-dimethylthiazole-2-yl)2,5-diphenyltetrazoliumbromide] assay (reagents purchased from MatTek). Viable tissues converted MTT into a blue formazan salt that was detected by measuring absorbance at a specific wavelength of light (A₅₇₀). Percent viability was calculated by comparing the A₅₇₀ reading of the test cultures to the A₅₇₀ of the untreated control culture, using the following formula: [Treated A₅₇₀/Untreated A₅₇₀]*100.

### Results

None of the concentrations tested produced any significant effects on cell viability (FIG. 2; Table 1). Cell viability was near 100% compared to the untreated control for all dilutions of Steamchol tested. These results show an advantageous affect of FABACs over retinoids which have been shown to be cytotoxic for fibroblasts and epithelial cells in the range of 0.6-3*10⁵ M (Varani et al. Journal of Investigative Dermatology (1993) 101, 839-842), and increase epithelial cell death (Ding et al. Invest Ophthalmol Vis Sci. 2013 Jun. 26; 54(6):4341-50).

TABLE 1
Cell Viability following a 24-hour Exposure to Steamchol

| Test compound | Cell Viability |
|---|---|
| Untreated Cells (Negative Control) | 100% |
| Positive control | 6.8% |
| DMSO vehicle | 101.0% |
| 2% Steamchol | 93.89% |
| 1% Steamchol | 96.88% |
| 0.1% Steamchol | 99.98% |
| 0.01% Steamchol | 100.41% |

### Example 2

### Effect of Steamchol or Aramchol on Cholesterol Efflux and ABCA1 mRNA and Protein Levels

Steamchol was incubated with human skin fibroblasts for 20 hours to measure cholesterol loading in the presence of [³H] cholesterol. After series of washing and adding efflux containing cholesterol acceptors, medium was collected and centrifuged and cell associated cholesterol was compared to effluxed cholesterol in the presence and absence of the similar FABAC. This research project also included quantification of mRNA of ABCA1 and direct measurement of ABCA1. Results demonstrated that cholesterol efflux in fibroblasts was significantly enhanced and that ABCA1 protein concentration increased approximately 2 fold when efflux took place in the presence of FABAC as compared to untreated cells.

Similarly, cells were pre-incubated with [³H] cholesterol, washed four times and then placed in an incubation medium with or without Aramchol for 20 hours. Following the incubation, radioactivity was measured separately in the medium and in the cells. Cholesterol efflux percentage was calculated as radioactivity in medium divided by the total radioactivity (cells+medium). Results demonstrated that cholesterol efflux in fibroblasts was significantly enhanced and that ABCA1 protein concentration increased approximately 2 fold when efflux took place in the presence of Aramchol as compared to untreated cells.

### Example 3

### Effect of Steamchol on Gene Expression in Skin Culture

A full-thickness in vitro skin culture model, Epiderm FT (MatTek, MA), was treated with Steamchol. Steamchol in DMSO (0.5%, 5000 ug/mL) was applied to the surface of each test culture and cells were collected 24 hours post-application. Control cells were similarly treated with DMSO without Steamchol.

Tissues were collected in RNA later for gene expression analysis. Gene expression was analyzed using validated Taqman gene expression assays in Taqman Low Density Array (TLDA) format. 94 genes that regulate a variety of known functions in skin were analyzed including the ABCA1 and SCD1 genes. The experimental set up was conducted in a 96-well format using validated Taqman gene expression assays. Each gene was assayed in duplicates. Statistics were carried out using the StatMiner software v4.2 (unpaired t-tests, p≦0.05, N=4) to compare the Steamchol group to the DMSO control group.

The effect of Steamchol on gene expression in the Epiderm FT culture revealed that out of the 94 selected genes in the panel, two genes, KRT 1 (keratin 1) and KRT 10 (keratin 10) demonstrated statistically significant deviation of more than two fold when compared to cells treated with DMSO (see Table 2 below). No significant change in mRNA levels of ABCA1 was observed. As shown in table 2, Steamchol significantly inhibits the expression of keratins 1 and 10 which are known markers for keratinocyte differentiation.

TABLE 2
Fold change of keratin 1 and 10 gene
expression after treatment with FABAC

| Gene ID | Gene Name | Fold Change |
|---|---|---|
| KRT10 | keratin 10 | −2.60 |
| KRT1 | keratin 1 | −2.49 |

In the basal layer of the epidermis where keratinocytes are mitotically active they express keratins 5 and 14. As the cell becomes suprabasal, keratins 1 and 10 are expressed while keratins 5 and 14 shut down. As the cells continue to move outward to the granular layer, they become filled with granules containing a variety of differentiation proteins; Loricin, Profilaggrin, Involucrin. Transgultaminase, an enzyme that cross-links keratins and other proteins into the impermeable cell envelop is also synthesized in this layer. Finally, the keratinocytes die and their dead, flattened squamous form composes the stratum corneum.

The effect on keratinocytes differentiation is thought to be reversible and recovery from cholesterol depletion is believed to occur when the stimulus is removed. Without wishing to be bound by any theory or mechanism of action, FABACs enhance the transport activity of ABCA1 protein, causing depletion in cholesterol levels at the lipid rafts, thereby reducing expression of keratins 1 and 10.

The activity of a fatty acid-bile acid conjugate was demonstrated herein, for the first time, to be similar to the effects known to be induced by retinoic acid. Both compounds affect differentiation markers at the spinous epidermal level and at the dermal extracellular matrix level. While retinoic acid acts via activation of nuclear receptor, a fatty acid-bile acid conjugate is presumed to induce organizational changes at the cell membrane level that lead to cascades similar to those of retinoic acid. In two different unrelated studies a fatty acid-bile acid conjugate was demonstrated to activate ABCA1 cell transporter and down regulate keratin 1 and 10 expression in human epidermal model. These two activities support reduction in keratinocytes differentiation, triggering a compensation mechanism at the epidermis and dermis levels and eventually leading to tissue rejuvenation, and possibly stronger extracellular matrix foundation that potentially leads to clinical manifestation of younger skin appearance.

Affecting cellular differentiation at the epidermal skin layers has been demonstrated to be the underlying mechanism for anti-aging activity. However, in many cases, such as in that of the retinoic acid, this activity comes with a “price” of serious adverse reactions such as teratogenicity, skin irritation and higher susceptibility to sun damage. This may be since the effect is initiated at the nuclear receptor levels and therefore is profound and slow to recover. The suggested mechanism of action of the FABACs of the invention provides a milder but yet promising biochemical path affecting the organization of lipids in the lipids rafts as a result of cholesterol depletion.

### Example 4

### Effect of FABACs on Keratinocyte Differentiation

In order to examine the effect of the disclosed FABACs on differentiation of keratinocytes, the highly differentiated full-thickness in vitro skin culture model Epiderm FT (MatTek, MA) is used. Duplicates of skin cultures are treated with three different concentrations of Steamchol in DMSO and three different concentrations of Aramchol in DMSO. One of the tested concentrations is 0.5% (5000 μg/mL) which was efficient in reducing gene expression of KRT1 and KRT10. Two non-treated skin cultures and two cultures treated with vehicle alone (DMSO) are used as negative controls. Two skin cultures treated with retinoic acid are used as a positive control.

The cells are collected 24 hours following application of the compositions to the surface of each test culture. Proteins are extracted from part the cells and subjected to Western Blot and ELISA analyses using primary antibodies specific for Keratin 1, Keratin 10, SCD1 and ABCA-1. Another part of the cells is fixated and subjected to immunohistochemical staining using primary antibodies specific for Keratin 1, Keratin 10, SCD1 and ABCA-1.

Down-regulation in expression of Keratin 1 and/or Keratin 10 is indicative of a decrease in keratinocyte differentiation.

### Example 5

### Effect of FABACs on Keratinocyte Proliferation and Expression of Extra Cellular Matrix (ECM) Proteins

In order to examine the effect of the disclosed FABACs on proliferation of keratinocytes and expression of ECM proteins, the highly differentiated full-thickness in vitro skin culture model Epiderm FT (MatTek, MA) is used.

Duplicates of skin cultures are treated with three different concentrations of Steamchol in DMSO and three different concentrations of Aramchol. One of the tested concentrations is 0.5% (5000 μg/mL) which was efficient in reducing gene expression of KRT1 and KRT10. Two non-treated skin cultures and two cultures treated with vehicle alone (DMSO) are used as negative controls. Two skin cultures treated with retinoic acid are used as a positive control.

The cells are collected 24 hours following application of the compositions to the surface of each test culture. Some of the cells are used for assaying cell proliferation using the MTT assay as used in Example 1 herein above. Proteins are extracted from part of the cells and subjected to ELISA analyses using primary antibodies specific for Elastin, Pro-Collagen and Matrix Metalloproteinase 1 (MMP-1). Another part of the cells is fixated and subjected to immunohistochemical staining using primary antibodies specific for Elastin, Pro-Collagen and Matrix Metalloproteinase 1 (MMP-1).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1.-37. (canceled)

38. A method of preventing or treating a skin condition related to aging comprising the step of administering to a subject in need thereof a topical composition comprising as an active ingredient a fatty acid bile acid conjugate (FABAC) and a dermatologically acceptable diluent or carrier, wherein the FABAC has the formula I:
W—X-G (I)
wherein G represents a bile acid or a bile salt radical; W represents one or two fatty acid radicals having 6-22 carbon atoms; and X represents a bonding member comprising a heteroatom or a direct C—C or C═C bond.

39. The method of claim 38, wherein the FABAC comprises two fatty acid radicals wherein at each occurrence W is independently a fatty acid radical having 6-22 carbon atoms; and X is independently a bonding member comprising a heteroatom or a direct C—C or C═C bond.

40. The method of claim 38, wherein said bonding member is selected from the group consisting of NH, P, S, O, or a direct C—C or C═C bond.

41. The method of claim 40, wherein said bonding member is NH.

42. The method of claim 38, wherein said one or two fatty acid radicals are independently selected from the group consisting of: stearic acid, behenic acid, arachidylic acid, palmitic acid, arachidonic acid, eicosapentaenoic acid, oleic acid.

43. The method of claim 42, wherein said one or two fatty acids is a stearic acid.

44. The method of claim 38, wherein said bile acid is selected from the group consisting of: cholic acid, ursodeoxycholic acid, chenodeoxycholic acid, deoxycholic acid, lithocholic acid, and derivatives thereof.

45. The method of claim 44, wherein said bile acid is a cholic acid.

46. The method of claim 38, wherein said FABAC is selected from the group consisting of:
3-beta-stearoyl-amido,7α,12α-dihydroxy-5-beta-cholan-24-oic acid,
3-beta arachidylamido,7α,12α-dihydroxy-5-beta-cholan-24-oic acid, and
a combination thereof.

47. The method of claim 46, wherein said FABAC is 3-beta-stearoyl-amido,7α,12α-dihydroxy-5-beta-cholan-24-oic acid.

48. The method of claim 38, wherein the skin condition related to aging is associated with at least one of chronological aging, photo-aging, skin atrophy or a combination thereof.

49. The method of claim 38, wherein the skin condition is selected from the group consisting of: fine lines, wrinkles, discoloration, uneven pigmentation, sagging, enlarged pores, rough skin, dry skin and stretch marks, uneven tone, blemishes, skin thickening or thinning and a combination thereof.

50. The method according to claim 49, wherein the skin condition is wrinkling.

51. The method according to claim 49, wherein the skin condition is fine lines.

52. The method of claim 38, wherein the composition is topically administered to a subject in need thereof.

53. A topical composition comprising as an active ingredient a fatty acid bile acid conjugate (FABAC) and a dermatologically acceptable diluent, carrier or excipient, wherein the FABAC has the formula I:
W—X-G (I)
wherein G represents a bile acid or a bile salt radical; W represents one or two fatty acid radicals having 6-22 carbon atoms; and X represents a bonding member comprising a heteroatom or a direct C—C or C═C bond.

54. The topical composition of claim 53, wherein said FABAC is 3-beta-stearoyl-amido,7α,12α-dihydroxy-5-beta-cholan-24-oic acid.

55. The topical composition of claim 53, formulated in a form selected from the group consisting of: aqueous solution, cream, lotion, water in oil or oil in water emulsion, multiple emulsion, silicone emulsion, microemulsion, nanoemulsion, foam, gel and an aqueous solution with a co-solvent.
